(19) United States
(12) Patent Application Publication
(10) Pub. No.: US 2014/0018316 A1
(43) Pub. Date: Jan. 16, 2014

(54) OPHTHALMIC PHARMACEUTICAL COMPOSITION

(71) Applicant: Hayashibara Co., Ltd., Okayama (JP)

(72) Inventors: Toshihiko Matsuo, Okayama (JP); Michio Kubota, Okayama (JP)

(21) Appl. No.: 14/029,332

(22) Filed: Sep. 17, 2013

Related U.S. Application Data
(62) Division of application No. 11/815,076, filed on Jul. 30, 2007.

(30) Foreign Application Priority Data
Jan. 28, 2005 (JP) 2005-021102

(51) Int. Cl. A61K 9/00; A61K 31/716

(52) U.S. Cl. CPC A61K 9/0048; A61K 31/716
USPC 514/61

(57) ABSTRACT

The present invention has an object to provide an ophthalmic pharmaceutical composition, particularly, an ophthalmic solution, ophthalmic ointment, ophthalmic wash, intraocular infusion solution, wash for anterior chamber, internal medicine, injection, or preservative for extracted cornea, which has improved therapeutic and/or prophylactic effects on the swelling, edema and walleye/cloudiness in the lens and/or the cornea induced by ophthalmic diseases including cataract and which is safe and successively used for a relatively long period of time. The present invention solves the above object by providing an ophthalmic pharmaceutical composition incorporated with a saccharide having a basic structure consisting of four glucose molecules bound together in a cyclic manner via a specific binding fashion.

### TECHNICAL FIELD

The present invention relates to a novel ophthalmic pharmaceutical composition for treating and/or preventing the swelling, edema or walleye/cloudiness in the lens and/or the cornea, which contains a saccharide having a basic cyclic-structure represented by General Formula 1 (designated as “a saccharide having a basic cyclic-structure”, hereinafter).

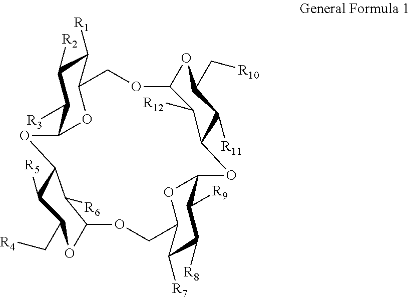

where R₁ through R₁₂ represent appropriate substituents.

### BACKGROUND ART

There have been known so far various types of ophthalmic diseases that induce the swelling, edema or walleye/cloudiness (may be called “nebula”) in the lens and/or the cornea due to internal or external factors. Among which, cataract is a serious disease that may cause blindness after its progressing. Such cataract can be roughly classified into congenital cataract and acquired cataract. Senile and diabetic cataracts, which are classified into acquired cataract, are diseases that are increased in incidence as the progress of ageing or life style related diseases; and it is speculated to be more and more increased in number of patients suffering from such diseases in the future of the society facing to that ageing society. Unfortunately, at present, almost no effective therapeutic treatment for cataract has been established, except for surgical treatment (ref. Nanzando Medical Dictionary, page 1,551, published in 1990 by Nanzando Co. Ltd., Tokyo, Japan). Even if such a surgical treatment has a problem that it could not be applied to patients under progressing diabetics or hypertension or it may induce post-surgery secondary cataract, where the posterior enclosing an intraocularly inserted lens may induce corneal walleye/cloudiness. Also surgical treatment would give a relatively large mental and economical burden on patients. Surgical treatment and symptomatic therapy used in combination with such surgical treatment will need medical admission and doctors' instructions and may further require devices, and this it will problematically restrict patients' usual social life styles due to such treatment.

While ophthalmic solutions are usually used in the ophthalmic field because of their readiness in portability and usability depending on requirement, as found in Japanese Patent Application Nos. 2002-348238 and 290830/98, ophthalmic solutions for treating cataract have been proposed. However, there are only few a that have been actually used, some of which would be problematic in safety or effectiveness. Thus, there has been strongly expected further development of ophthalmic compositions for treating and preventing the swelling, edema or walleye/cloudiness in the lens and/or the cornea of the eye. International Patent Publication No. WO 2004/020,552 discloses an ophthalmic solution incorporating a non-reducing saccharide composed of four glucose molecules bound together in a cyclic manner via the α-1,3 and α-1,6 linkages, where all the substituents of a saccharide having a basic cyclic-structure are hydroxyl groups, i.e., cyclo{→6)-α-D-glucopyranosyl-(1→3)-α-D-glucopyranosyl-(1→6)-α-D-glucopyranosyl-(1→3)-α-D-glucopyranosyl-(1→} (simply designated as “cyclic tetrasaccharide”, hereinafter).

The above ophthalmic solution incorporated with cyclic tetrasaccharide as disclosed in International Patent Publication No. WO 2004/020,552 is, however, this cyclic tetrasaccharide is disclosed to treat ophthalmic inflammation and dry eye, and the specification neither discloses nor suggests any ophthalmic pharmaceutical composition for treating and/or preventing the swelling, edema or walleye/cloudiness in the lens and/or the cornea.

### DISCLOSURE OF INVENTION

The present invention has an object to provide an ophthalmic pharmaceutical composition, which has improved therapeutic and/or prophylactic effects on the swelling, edema or walleye/cloudiness induced in the lens and/or the cornea when subjects are suffering from ophthalmic diseases such as cataract, and which has an advantageous usefulness and a relatively high safety even when successively used for a relatively long period of time. More particularly, the object is to provide an ophthalmic solution, ophthalmic ointment, ophthalmic wash, intraocular infusion solution, wash for anterior chamber, internal medicine, injection, and preservative for extracted cornea.

Considering these circumstances, the present inventors screened ingredients that have improved therapeutic and/or prophylactic effects on the swelling, edema or walleye/cloudiness induced in the lens and/or the cornea when subjects are suffering from ophthalmic diseases such as cataract, and have a relatively high safety even when administered to subjects for a relatively long period of time. They unexpectedly found that a saccharide having a basic cyclic-structure exerts distinct therapeutic and/or prophylactic effects on symptoms of the swelling, edema or walleye/cloudiness induced in the lens and/or the cornea without causing side effects even when administered for a relatively long period of time. Thus, they accomplished this invention. The present invention solves the above object by providing an ophthalmic pharmaceutical composition containing such a saccharide having a basic cyclic-structure.

### BEST MODE FOR CARRYING OUT THE INVENTION

The basic saccharide having a basic cyclic-structure used in the present invention is a saccharide having a structure represented by General Formula 1, which includes a cyclic tetrasaccharide where all the substituents therein are hydroxyl groups, those which are obtained by replacing one or more of the hydroxyl groups of the cyclic tetrasaccharide with substituents other than hydroxyl groups (designated as “derivatives of cyclic tetrasaccharide”, hereinafter), and others obtained by introducing appropriate substituents into the cyclic tetrasaccharide through reactions such as glycosylation, esterification, etherification, sulfonylation, and ammination. One or more of these cyclic tetrasaccharide and derivatives thereof can be used in an appropriate combination and incorporated into the ophthalmic pharmaceutical composition of the present invention. Any cyclic tetrasaccharide and derivatives thereof can be used independently of their origins and processes, and those which are prepared by fermentation methods, enzymatic methods, and organic synthetic methods can be used. Reaction solutions obtained by the above methods can be freelyused intact or after concentrated, partially purified, or highly purified.

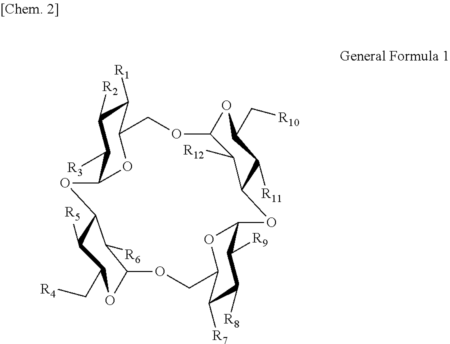

where R₁ through R₁₂ represent appropriate substituents.

The cyclic tetrasaccharide used in the present invention can be prepared by enzymatic methods using as materials amylaceous substances or saccharides originated therefrom; a method of converting panose into cyclic tetrasaccharide by using α-isomaltosyl-transferring enzyme, disclosed in International Patent Publication No. 01/90,338; or a method of preparing cyclic tetrasaccharide directly from starch using α-isomaltosylglucosaccharide-forming enzyme and α-isomaltosyl-transferring enzyme in combination, disclosed in International Patent Publication No. 02/10,361. These methods can be advantageously, industrially employed because they use abundant, low cost amylaceous substances as materials to facilitate the production of cyclic tetrasaccharide in a relatively high yield and at a relatively low cost. Cyclic tetrasaccharide has an anhydrous amorphous form, anhydrous crystalline form, crystalline monohydrate form, and crystalline pentahydrate form, and any of which can be used in the present invention. Among which, those in an anhydrous crystalline form, crystalline monohydrate form, and anhydrous amorphous form have a distinct dehydrating ability, and they also function as desiccants in preparing ophthalmic pharmaceutical compositions containing cyclic tetrasaccharide, in the form of a solid such as a powder or granule used after dissolved when in use.

Examples of the derivatives of cyclic tetrasaccharide used in the present invention can be illustrated with saccharides introduced with at least one among one or more types of glycosyl groups such as α-D-glucopyranosyl group, β-D-galactopyranosyl group, and β-D-chitosaminyl group (may be designated as “branched cyclic-tetrasaccharide”, hereinafter). These saccharides can be prepared by allowing the above-identified α-isomaltosylglucosaccharide-forming enzyme and α-isomaltosyl-transferring enzyme in combination to act on starch. Also the above saccharides can be advantageously prepared by allowing one or more enzymes having a saccharide-transferring ability, such as cyclomaltodextrin glucanotransferase, β-galactosidase, α-galactosidase, and lysozyme, to act on cyclic tetrasaccharide in the presence of monosaccharides, oligosaccharides and/or polysaccharides usable as substrates for the above enzymes by a method such as the one disclosed in International Patent Publication No. WO 02/072,594.

Examples of the derivatives of cyclic tetrasaccharide other than the above-identified ones, usable in the present invention, can be illustrated with cyclic tetrasaccharide or branched cyclic tetrasaccharides where at least one or two selected from among one or more types of substituents from appropriate substituents such as those which have hydrocarbon groups, substituents with oxygen excluding hydroxyl group, substituents with nitrogen, substituents with sulfur, or substituents with halogen; and include “saccharide derivatives” disclosed in Japanese Patent Kokai No. 2003-160595. These derivatives of cyclic tetrasaccharide can be prepared in usual manner by dissolving, suspending or soaking cyclic tetrasaccharide and/or branched cyclic tetrasaccharide in an appropriate solvent, and optionally adding a catalyst in combination with a reactive reagent as a donor for substituent to the resulting mixture, and allowing the mixture to stand at appropriate reaction conditions of temperature, time, pH, pressure, etc., under mixing or stirring conditions by using an appropriate method. The formed saccharide derivatives can be purified by removing the remaining intact reactive reagent, solvent and/or catalyst using appropriate purification methods.

Since the ophthalmic pharmaceutical composition of the present invention such as an ophthalmic solution, ophthalmic ointment, ophthalmic wash, intraocular infusion solution, wash for anterior chamber, internal medicine, injection, or preservative for extracted cornea directly contacts with ophthalmic mucosae, impurities such as pyrogens contained in the saccharides having a basic cyclic-structure to be incorporated into the ophthalmic pharmaceutical composition should preferably be removed by purification methods such as treatments with activated carbon, ion-exchange chromatography, gel filtration chromatography, and membrane filtration.

The saccharides having a basic cyclic-structure used in the present invention can be those which consist of one or more types of them; those which contain both any of the saccharides having a basic cyclic-structure and a saccharide(s) other than the saccharides having a basic cyclic-structure, such as glucose, isomaltose, maltose, maltotriose, and maltodextrin, which coexist with the saccharides having a basic cyclic-structure in their preparation; or those which are obtained by converting the above reducing saccharides into sugar alcohols by hydrogenation. While in case that the ophthalmic pharmaceutical composition of the present invention contains a substance having an amino group intramolecularly, such as amino acids, the coexisting reducing-saccharides including glucose may cause a particular problem of deteriorating the quality of effective ingredients in the composition and/or of the composition per se and therefore desired saccharides to be incorporated are those which have a lower content of reducing saccharides that cause the Maillard reaction. Because of this, the saccharides, which contain a saccharide having a basic cyclic-structure, to be incorporated into the ophthalmic pharmaceutical composition of the present invention are preferably those which contain such a saccharide having a basic cyclic-structure in an amount of at least 98% by weight (throughout the specification, the term “% by weight” is simply abbreviated as “%”, unless specified otherwise), preferably, at least 99%, and more preferably, at least 99.5%; or it can be used those which are prepared by hydrogenating reducing saccharides, which coexist with the saccharide having a basic cyclic-structure, to lower their reducibility.

The method of incorporating a saccharide having a basic cyclic-structure into an ophthalmic pharmaceutical composition is not specifically restricted and such a saccharide can be incorporated thereunto at any appropriate step ranging from the material stage through the final product stage depending on the form of the composition, or incorporated into a ready-made product using any one or more methods appropriately selected from the group consisting of mixing, kneading, dissolving, melting, dispersing, suspending, emulsifying, soaking, penetrating, spreading, applying, coating, spraying, injecting, crystallizing, and solidifying. The form of the saccharide having a basic cyclic-structure used in the present invention should not specifically be restricted, and it can be appropriately selected from, for example, a solution, syrup, massecuite, solid, and powder form.

The administration of the ophthalmic pharmaceutical composition containing the saccharide(s) having a basic cyclic-structure of the present invention to patients with cataract, those with walleye/cloudiness in the lens and/or the cornea, observed when they are in surgery for treating cataract or the lens, or to those with cloudiness in the posterior enclosing the lens after surgery inhibits the progress of cloudiness in the patients' lens and/or cornea and posterior, or even improves the corneal cloudiness, and it can also prevent the onset of such symptoms when administered to subjects before observation of such symptoms or before such surgery. Since the ophthalmic pharmaceutical composition of the present invention has an action of inhibiting the swelling of the lens and the corneal edema, it can be used as an ophthalmic solution, ophthalmic wash, ophthalmic ointment, intraocular infusion solution, wash for anterior chamber, internal medicine, injection, and preservative for extracted cornea to improve the swelling or edema of the lens or the cornea and to prevent the edema or walleye/cloudiness of extracted cornea during its transplantation.

The ophthalmic pharmaceutical composition of the present invention can be prepared by using conventional bases depending on its form and incorporating any of the saccharides having a basic cyclic-structure into the bases. The bases used for an ophthalmic solution, ophthalmic wash, intraocular infusion solution, wash for anterior chamber, injection, and preservative for extracted cornea include any of those which are used in conventional ophthalmic pharmaceutical compositions; and usually refined water containing electrolytes can be used. The ophthalmic pharmaceutical composition can be prepared in the form of a solution prior to use or prepared into a solid form for use after dissolved when in use. In the case of the latter form, it can be used after dissolved in refined water, physiological saline, etc. Examples of the form of the composition include tablets, granules, powders, etc. In preparing the composition in an ointment form, an ophthalmic petrolatum, etc., is used. The above preparations can be prepared in accordance with conventional methods, and in any case such preparations can desirably be sterilized by using conventional means such as membrane filter, autoclave, etc. Since the ophthalmic pharmaceutical composition of the present invention directly contacts with the ophthalmic mucosae, it should preferably be removed pyrogen by an appropriate means to be substantially free of pyrogen.

The content of the saccharide having a basic cyclic-structure in the ophthalmic pharmaceutical composition of the present invention should not specifically be restricted as long as it can exert the above-identified physiological action, and a desired content is usually at least 0.01% by weight (throughout the specification the term “% by weight” is abbreviated as “%” hereinafter, unless specified otherwise) or about 0.01 to 30% to the total amount of the ophthalmic pharmaceutical composition. Considering both the influence on ophthalmic mucosae and the therapeutic and/or prophylactic effects on cataract, the saccharide content is preferably 0.5 to 20%, and more preferably, 4 to 15%. Particularly, in the case of conducting a treatment such as surgery that may possibly injure the lens, a possibly injured site of the cornea or the lens and the cloudiness in the lens inducible thereby and its progress can be lowered as much as possible by using the ophthalmic pharmaceutical composition containing at least 5% of the saccharide(s) having a basic cyclic-structure prior to such treatment. In terms of the inhibition of the swelling of the lens, the saccharide content is desirably at least 0.1%. Since the ophthalmic pharmaceutical composition of the present invention directly contacts with ophthalmic mucosae, the pH of the composition is desirably a pH at around neutral pH, particularly, a pH of 6.5 to 7.5. The osmotic pressure of the composition should desirable be controlled to give a compression ratio of about 0.5 to about 4.0, preferably, about 1.0 to about 1.5. To control the pH and the osmotic pressure of the composition, conventional methods can be used. The term “compression ratio” as referred to as in the present invention means a value of dividing the osmotic pressure of the ophthalmic pharmaceutical composition of the present invention by that of the human tear fluid.

Among saccharides, the saccharides having a basic cyclic-structure are so stable as not to substantially induce amino carboxylation reaction even when in combination with vitamins, peptides, proteins, etc., having amino groups; and they also have a property of stabilizing the above ingredients. Accordingly, the ophthalmic pharmaceutical composition of the present invention can be optionally prepared by being appropriately incorporated with one or more ingredients of pharmaceutically acceptable electrolytes, amino acids, vitamins and derivatives thereof, lipids, reducing and non-reducing saccharides other than the saccharide having a basic cyclic-structure, sugar alcohols, water-soluble polysaccharides, inorganic salts, emulsifiers, antioxidants, substances with chelating action, antibiotics, anti-inflamatories, therapeutic agents for cataract, agents for preparing pharmaceuticals other than the above-identified materials, and pharmaceuticals.

Explaining concretely the ingredients other than the saccharides having a basic cyclic-structure used in the ophthalmic pharmaceutical composition of the present invention, they are, for example, those which have a therapeutic effect on cataract such as glutathione, hormone of salivary gland, tiopronin, pirenoxine; saccharides such as glucose, trehalose (α,α-trehalose, α,β-trehalose, β,β-trehalose), and maltose; oligosaccharides; sugar alcohols such as mannitol and sorbitol; electrolytes such as sodium chloride, sodium hydrogenphosphate, potassium chloride, magnesium sulfate, and calcium chloride; amino acids such as glycine and alanine; and vitamins such as thiamin hydrochloride, riboflavin sodium phosphate, pyridoxine hydrochloride, nicotinic acid amide, folic acid, biotin, vitamin A,L-ascorbic acid, and L-ascorbic acid 2-glucoside, and derivatives thereof; one or more of which can be appropriately incorporated in combination into the ophthalmic pharmaceutical composition. Among the above ingredients, since L-ascorbic acid 2-glucoside has a distinct ability of stably storing organs for transplantation and an advantageous inhibitory action on the radical formation, it can effectively enhance the above-identified physiological action inherent to the saccharides having a basic cyclic-structure when used with any of them.

In particular, when the ophthalmic pharmaceutical composition of the present invention is in the form of an ophthalmic solution, it can be incorporated in an appropriate combination with one or more ingredients effective for treating/preventing the above-identified cataract, etc., and additives for preparations used in conventional ophthalmic preparations; preservatives such as water, alcohols, p-oxymethyl benzoate, sodium dehydroacetate, and benzalkonium chloride; buffers such as borax, boric acid, and sodium hydrogencarbonate; thickeners such as methyl cellulose, carboxymethyl cellulose, chondroitin sulfate, poly(vinyl alcohol), and pullulan; solubilizing agents such as polysorbate 80; and stabilizers such as sodium edetate and sodium hydrogensulfite.

When the ophthalmic pharmaceutical composition of the present invention is in the form of an ophthalmic ointment, it can be prepared with conventional bases for ointments; ophthalmic white petrolatum and sodium hydrogensulfite. Liquid paraffin, etc., can be used as a pharmaceutical additive.

The use and dose of the ophthalmic pharmaceutical composition of the present invention can be appropriately controlled depending on the symptom of diseases and the degree of surgery. In the case that the ophthalmic pharmaceutical composition of the present invention is the form of an ophthalmic solution, it is usually administered at a dose of one to four drops per shot (about 0.025 to about 0.1 ml) and at a frequency of 1 to about 10 times a day. In the case that the ophthalmic pharmaceutical composition of the present invention is the form of an ophthalmic wash, it is usually used to wash users' eyes in such a manner of placing a specific container with about five milliliters of the composition to closely attach it to the face line around the eyes, allowing to bend users' heads backward to look up, and allowing the users' eyes to blink several times in the ophthalmic wash usually at a frequency of one to about six times a day; or washing the users' eyes one to five times a day with about one to five milliliters of the ophthalmic wash by using a washing bottle, etc. In the case that the ophthalmic pharmaceutical composition of the present invention is in the form of an ophthalmic ointment, it is usually applied to the inside of the conjunctival sac in an appropriate amount usually at a frequency of one to three times a day.

The ophthalmic pharmaceutical composition of the present invention can be advantageously used in preventing and/or treating walleye/cloudiness in the cornea, cloudiness in the lens, swelling of the lens, edema of the cornea that are occurred in animals including mammals, fowls, reptiles, amphibia, and fishes, as well as humans.

The term “cataract” as referred to as in the present invention means a disease that exhibits symptoms of causing cloudiness on the surface and/or the inside of the lens or inducing the swelling of the lens, and it includes both congenital cataract and acquired cataract (cf. “Great Medical Dictionary”, page 1,551, 1990, published by Nanzando Co., Ltd., Tokyo, Japan). Concrete examples of such are congenital cataract such as congenital pseudo-cataract, congenital membrane cataract, congenital coronary cataract, congenital lamellar cataract, congenital punctate cataract, and congenital filamentary cataract; and acquired cataract such as geriatric cataract, secondary cataract, browning cataract, complicated cataract, diabetic cataract, traumatic cataract, and others inducible by electric shock, radiation, ultrasonic, drugs, systemic diseases, and nutritional disorders. Acquired cataract further includes postoperative cataract with symptoms of causing cloudiness in the posterior encapsulating a lens inserted to treat cataract. Examples of diseases that develop edema include bullous keratopathy.

The ophthalmic pharmaceutical composition, incorporated with the saccharide(s) having a basic cyclic-structure, according to the present invention for treating and/or preventing ophthalmic diseases that induce swelling, edema or walleye/cloudiness in the lens and/or the cornea is explained with reference to the following examples but it should not limit the scope of the present invention.

### Experiment 1

### Influence of Cyclic Tetrasaccharide on Swelling and Cloudiness in the Lens

Using an injured pig lens employed as a model for traumatic cataract, the influence of cyclic tetrasaccharide on the cloudiness in the lens was experimented as follows: A test solution was previously prepared by dissolving a cyclic tetrasaccharide crystal, penta hydrate, which had been prepared by the method in the later described Reference for Example 2, in physiological saline into a physiological saline containing 100 mM cyclic tetrasaccharide. As a positive control, a physiological saline containing 100 mM α,α-trehalose, a reagent grade specimen, commercialized by Hayashibara Biochemical Laboratories, Inc., Okayama, Japan, was used; andasanegativecontrol, physiological saline was used. Two milliliter aliquots of any one of the above three kinds of aqueous solutions were added to every two wells in respective commercialized 24-well culture plates. In one of the two wells containing the same kind of solution in each plate was placed a pig lens, which had been obtained from a slaughter-house, extracted by incision within three hours after the obtention and without injuring, and washing the extracted lens with physiological saline; while in the other of the two wells was placed a pig lens, which had been prepared by the same procedure as in the above but injured with a 27-gauge injection needle (commercialized by Terumo Co., Tokyo, Japan) to form an injured line, about one millimeter in length. These lens were allowed to stand at ambient temperature and macroscopically and microscopically observed their change in the degree of cloudiness and in the shape of swelling, etc. This experiment was run in a triplicate manner, and the macroscopic and microscopic observations were done just after (0), 1 hour, 2 hours, 6 hours, and 24 hours after the lenses were placed in the wells, and then observed daily over 28 days. A similar experiment was further conducted using a physiological saline containing 75 mM, 50 mM, 20 mM, 10 mM, or 1 mM cyclic tetrasaccharide and a lens that had not been injured with the injection needle. The degree of cloudiness in each lens, observed with “STEREOMICROSCOPE SZX12”, a product name of a stereomicroscope commercialized by Olympus Co., Tokyo, Japan, was expressed by photographing a transmitted light image of each lens with “PENGUIN 150CL”, a digital camera commercialized by Pixela Co., Tokyo, Japan, capturing the transmitted image by a computer using a view finder commercialized by Olympus Co., Tokyo, Japan, and expressing numerically the degree of light and shade of the captured image as a gray-scale using “Scion Image for Windows Beta 4.0.2”, a software commercialized by Scion Co., Maryland, U.S.A., to obtain a mean density for each lens. The time course change in the degree of cloudiness in each lens placed in each solution was expressed with a relative value, regarding the degree of cloudiness, just after the lens free of injury had been placed in physiological saline, as 10. The change of cloudiness and swelling are respectively in Tables 1 and 2. In Table 1, there show only the results from time 0 hour through two days and from days 7, 14 and 28.

TABLE 1

| | | Time after soaking lenses in test solutions or control solutions | | | | | | | | |
|---|---|---|---|---|---|---|---|---|---|---|
| Test groups | Injury | 0 | 1 hour | 2 hours | 6 hours | 24 hours | 2 days | 7 days | 14 days | 28 days |
| Negative control | No | 10 | 11 | 12 | 12 | 12 | 15 | 56 | 72 | 90 |
| solution | Yes | 10 | 12 | 19 | 31 | 90 | 92 | ND | ND | ND |
| Test solution | No | 10 | 13 | 12 | 12 | 12 | 11 | 12 | 15 | 15 |
| (100 mM) | Yes | 10 | 10 | 10 | 12 | 12 | 12 | ND | ND | ND |
| Test solution | No | 10 | 11 | 12 | 12 | 12 | 12 | 14 | 21 | 29 |
| (75 mM) | | | | | | | | | | |
| Test solution | No | 10 | 11 | 12 | 12 | 12 | 13 | 15 | 20 | 28 |
| (50 mM) | | | | | | | | | | |
| Test solution | No | 10 | 11 | 12 | 12 | 12 | 14 | 51 | 60 | 71 |
| (20 mM) | | | | | | | | | | |
| Test solution | No | 10 | 12 | 11 | 12 | 11 | 13 | 49 | 59 | 87 |
| (10 mM) | | | | | | | | | | |
| Test solution | No | 10 | 11 | 12 | 11 | 12 | 14 | 55 | 61 | 85 |
| (1 mM) | | | | | | | | | | |
| Positive control | No | 10 | 13 | 13 | 12 | 14 | 19 | 36 | 39 | 75 |
| solution | Yes | 10 | 10 | 10 | 15 | 46 | 89 | ND | ND | ND |

Note:
The symbol “ND” means that the degree of cloudiness was not measured.

TABLE 2

| | | Time after soaking lenses in test solutions or control solutions | | | | | | | | |
|---|---|---|---|---|---|---|---|---|---|---|
| Test groups | Injury | 0 | 1 hour | 2 hours | 6 hours | 24 hours | 2 days | 7 days | 14 days | 28 days |
| Negative control | No | — | — | — | — | — | — | — | ◯ | ◯ |
| solution | Yes | — | — | — | — | ◯ | ◯ | ND | ND | ND |
| Test solution | No | — | — | — | — | — | — | — | — | — |
| (100 mM) | Yes | — | — | — | — | — | — | ND | ND | ND |
| Test solution | No | — | — | — | — | — | — | — | — | — |
| (75 mM) | | | | | | | | | | |
| Test solution | No | — | — | — | — | — | — | — | — | — |
| (50 mM) | | | | | | | | | | |
| Test solution | No | — | — | — | — | — | — | — | — | ◯ |
| (20 mM) | | | | | | | | | | |
| Test solution | No | — | — | — | — | — | — | — | — | ◯ |
| (10 mM) | | | | | | | | | | |
| Test solution | No | — | — | — | — | — | — | — | — | ◯ |
| (1 mM) | | | | | | | | | | |
| Positive control | No | — | — | — | — | — | — | — | — | ◯ |
| solution | Yes | — | — | — | — | — | ◯ | ND | ND | ND |

Note:
The symbols “◯”, “—” and “ND” mean that “swelling was observed”, “no swelling was observed”, and “no observation was done for swelling”, respectively.

As evident from Table 1, on 7 days after initiating observations, the lenses with no injury, which had been soaked in physiological saline as a negative control solution and a physiological saline containing α,α-trehalose used as a positive control solution, were confirmed to have increased degrees of cloudiness of 56 and 36, respectively, meaning that their cloudiness had progressed. As evident from Table 2, on 28 days the swelling of the lenses was macroscopically observed in addition to their cloudiness. In the case of using the positive control solution, the cloudiness in the lens was inhibited compared with that using the negative control solution. The lenses with no injury, which had been soaked in the test solutions containing cyclic tetrasaccharide, still retained their transparency at a degree of cloudiness of 15 and gave no swelling even on 28 days after initiating their observations. The lenses which had been injured with the injection needle were macroscopically observed to exhibit cloudiness in their injured sites just after their injury, and when soaked in the negative control solution and the positive control solution, the lenses exhibited an increased degree of cloudiness as high as 90 or 46 and were observed to have cloudiness throughout them at 24 hours after initiating their observations. The lenses, which had been soaked in the negative control solution were observed to have swelling from 24 hours after initiating their observations, and the one which had been soaked in the positive control solution was observed to have swelling from 2 days after initiating its observation. The lens, which had been soaked in the test solution, exhibited a partial cloudiness only at the injured site that did not spread throughout the lens and stayed only to show a partial cloudiness, without giving a distinctly increased degree of cloudiness in the lens even after 2 days from initiation of its observation. Even on 2 days after initiating observation, the swelling of the lens was not observed. In the case of using both the lenses with no injury and a physiological saline containing 75 mM or 50 mM cyclic tetrasaccharide, similarly as in the case of using a physiological saline containing 100 mM cyclic tetrasaccharide, they exhibited a degree of cloudiness of 29 or 28 even at 28 days after initiating their observations and did not exhibit distinctly increased cloudiness from initiation of their observations. No swelling was observed until 28 days after initiating their observations. In the case of using both the lenses with no injury and a physiological saline containing 20 mM, 10 mM or 1 mM cyclic tetrasaccharide, cloudiness was observed throughout the lenses from seven days after initiating their observations. All the degrees of cloudiness in the above cases were inhibited to an extent lower than that with the negative control solution but higher than that with the positive control solution. No swelling of the lenses was observed until 14 days after initiating their observations.

The experiment results indicate that cyclic tetrasaccharide at a concentration of 50 to 100 mM or higher has an action of inhibiting the swelling and cloudiness in the lens and maintaining the transparency of the lens and/or has an action of inhibiting the swelling of the lens and the spreading of cloudiness in the lens; and in view of the durability of such effects, it is preferably used at concentrations of 50 mM or higher, particularly, a concentration of 100 mM. It also indicates that cyclic tetrasaccharide has a satisfactory swelling-inhibitory-effect on the lens at concentrations of 1 mM or higher.

### Experiment 2

### Influence of Cyclic Tetrasaccharide on Corneal Edema and Walleye/Cloudiness

Experiment to examine the influence of cyclic tetrasaccharide on corneal walleye/cloudiness was conducted as follows: A physiological saline containing 100 mM cyclic tetrasaccharide was previously prepared for use as a test solution by dissolving a cyclic tetrasaccharide crystal, pentahydrate, which had been prepared by the later described method in Example for Reference 2. As a positive control solution, it was prepared a physiological saline containing 100 mM α,α-trehalose, a reagent grade specimen, commercialized by Hayashibara Biochemical Laboratories, Inc., Okayama, Japan; and as a negative control solution, physiological saline was used. Two milliliters of any one of the above three kinds of aqueous solutions were added to respective two wells in a commercialized 24-well culture plate. To each well in each culture plate a circular corneal fragment, which had been extracted from a pig eye ball obtained from a slaughterhouse by using a trephine for corneal transplantation, was placed and macroscopically and microscopically observed for changing in both the degree of corneal walleye/cloudiness and the change of corneal shape such as corneal edema. The macroscopic and microscopic observations were done just after the corneas were placed in the wells at (0) and at 1, 2, 6, 24, 48 and 72 hours after their placing. The methods of macroscopic and microscopic observations and the analysis of the time course change of corneal walleye/cloudiness were conducted by the same ones as used in Experiment 1. Tables 3 and 4 show the transition of corneal walleye/cloudiness and that of the corneal edema, respectively.

TABLE 3

| | Time after soaking the cornea in test | | | | | | |
|---|---|---|---|---|---|---|---|
| | solution or control solutions | | | | | | |
| | | | | | 24 | 48 | 72 |
| Test groups | 0 | 1 hour | 2 hours | 6 hours | hours | hours | hours |
| Negative control | 10 | 12 | 12 | 21 | 45 | 80 | 80 |
| solution | | | | | | | |
| Test solution | 10 | 11 | 11 | 13 | 16 | 16 | 19 |
| (100 mM) | | | | | | | |
| Positive control | 10 | 11 | 11 | 13 | 30 | 60 | 70 |
| solution | | | | | | | |

TABLE 4

| | Time after soaking the cornea in test | | | | | | |
|---|---|---|---|---|---|---|---|
| | solution or control solutions | | | | | | |
| | | | | | 24 | 48 | 72 |
| Test groups | 0 | 1 hour | 2 hours | 6 hours | hours | hours | hours |
| Negative control | — | — | — | — | ◯ | ◯ | ◯ |
| solution | | | | | | | |
| Test solution | — | — | — | — | — | — | — |
| (100 mM) | | | | | | | |
| Positive control | — | — | — | — | ◯ | ◯ | ◯ |
| Solution | | | | | | | |

Note
“◯”: Corneal edema was observed.
“—”: No corneal edema was observed.

As evident from the results in Tables 3 and 4, the corneas, which had been soaked in the physiological saline as negative control solution or the physiological saline containing α,α-trehalose as positive control solution, gave an increased degree of walleye/cloudiness as high as 45 or 30 at 24 hours after the initiation of their observations, resulting in an observation of corneal walleye/cloudiness and also macroscopic corneal edema. While the cornea, which had been soaked in the test solution containing cyclic tetrasaccharide, gave no distinct difference in walleye/cloudiness and exhibited a degree of walleye/cloudiness of 19 even at 72 hours after initiating its observation while retaining its transparency. No occurrence of corneal edema was observed.

The experimental results indicate that cyclic tetrasaccharide has actions of inhibiting corneal walleye/cloudiness and maintaining its transparency, as well as inhibiting the occurrence of corneal edema; and ophthalmic pharmaceutical compositions such as ophthalmic solutions containing cyclic tetrasaccharide can be used as inhibitors for corneal walleye/cloudiness and edema and used as preservatives of corneas for transplantation.

### Example for Reference 1

### Example for Preparation of Cyclic Tetrasaccharide

In accordance with the method in Example A-1 disclosed in International Patent Publication No. WO 02/10,361, it was prepared from starch a syrup containing a mixture of cyclic tetrasaccharide and branched cyclic tetrasaccharide, having a concentration of 80% and containing 0.6% of glucose, 1.5% of isomaltose, 12.3% of maltose, 63.5% of cyclic tetrasaccharide, 5.2% of saccharide derivative of cyclic tetrasaccharide, and 16.9% of other saccharides. The product can be used in preparing ophthalmic pharmaceutical compositions for treating ophthalmic diseases including cataract, that induce swelling, edema or walleye/cloudiness in the lens and/or the cornea.

### Example for Reference 2

### Example for Preparation of Cyclic Tetrasaccharide

In accordance with the method in Example A-3 disclosed in International Patent Publication No. WO 02/10,361, the cyclic tetrasaccharide was prepared from material tapioca starch a syrup containing a mixture of cyclic tetrasaccharide and derivatives thereof, followed by subjecting the syrup to purification, concentration, and drying and crystallizing to obtain a cyclic tetrasaccharide crystal, pentahydrate, with a purity of 99.6%, in accordance with the methods in Examples A-6 and A-7 disclosed in International Patent Publication No. WO 02/10,361, now U.S. Pat. No. 7,241,606. The product can be used in preparing ophthalmic pharmaceutical compositions for treating ophthalmic diseases including cataract, that induce swelling, edema or walleye/cloudiness in the lens and/or the cornea.

In accordance with the method in Experiment 31 or 32 disclosed in International Patent Publication No. WO 02/10,361, the above cyclic tetrasaccharide crystal, pentahydrate, was further dried into a powdery cyclic tetrasaccharide crystal, monohydrate, and a powdery anhydrous crystalline cyclic tetrasaccharide. Similarly as in a powdery cyclic tetrasaccharide crystal, pentahydrate, the above cyclic tetrasaccharides can be used in preparing ophthalmic pharmaceutical compositions for treating ophthalmic diseases including cataract, that induce walleye/cloudiness in the lens and/or the cornea. Also the above cyclic tetrasaccharides can be advantageously used as a pulverization base for pulverizing ophthalmic pharmaceutical compositions to be used after dissolving when in use and used for treating ophthalmic diseases that induce swelling, edema or walleye/cloudiness in the lens and/or the cornea, under the normal conditions of temperature and pressure.

### Example for Reference 3

### Preparation of Branched Cyclic Tetrasaccharide

In accordance with the method in Experiment 4-4 (a) in International Patent Publication No. WO 02/072,594, 20 g of cyclic tetrasaccharide crystal, pentahydrate, prepared in Example for Reference 2, and 20 g of lactose, a special grade specimen commercialized by Wako Pure Chemical Industries, Tokyo, Japan, were dissolved in 93.3 g of 20 mM sodium acetate buffer (pH 6.0). To the solution was added 3 units/g lactose of “BIOLACTANE 5”, a β-galactosidase specimen of Bacillus circulans produced by Daiwa Fine Chemical Co., Ltd., Tokyo, Japan, followed by reacting the mixture at 40° C. for 24 hours and boiling the reaction mixture for 20 min to inactivate the remaining enzyme. The resulting solution was in usual manner purified, decolored, and concentrated, and then the concentrate was admixed with 4.8 g of sodium hydroxide and retained at 100° C. for one hour to decompose reducing sugars. The reaction solution was in usual manner desalted, filtered, and concentrated, and the concentrate was fed to “YMC-Pack ODS-AQR355-15AQ, S-10/20 μm, 120A”, a preparative liquid chromatography produced by YMC Co. Ltd., Tokyo, Japan, to collect fractions containing a saccharide having a basic cyclic-structure and a purity of at least 97%, using refined water as a moving phase. The collected fractions were in usual manner decolored, concentrated, and spray-dried to obtain an amorphous powder. Methods such as NMR measurement revealed that the product was a non-reducing branched cyclic tetrasaccharide having a structure where one mole of D-galactose molecule, as a substituent, bound to cyclic tetrasaccharide. The product can be used in preparing ophthalmic pharmaceutical compositions for treating ophthalmic diseases including cataract, that induce swelling, edema or walleye/cloudiness of the lens and/or the cornea.

### Example for Reference 4

Preparation of Saccharide having a Basic Cyclic-Structure with Methyl Group as Substituent

Five parts by weight of an anhydrous crystalline cyclic tetrasaccharide prepared by the method in Example for Reference 2 was dissolved in 125 parts by weight of anhydrous dimethylsulfoxide and then admixed with 12.5 parts by weight of sodium hydroxide, followed by mixing the mixture, cooling it in an ice bath for 10 min, and then heating it at 60° C. for two hours. To the resulting mixture 22.5 parts by weight of methyl iodide was gradually added under ice cooling conditions and then the mixture was stirred at ambient temperature for 18 hours, admixed with 40 parts by weight of methanol, and further admixed with 200 parts by weight of ice-cooled distilled water. To the resulting mixture was added 500 parts by weight of chloroform before mixing, followed by allowing the mixture to stand until it separated into a water phase and a chloroform phase and then collecting the chloroform phase. Fifty parts by weight of distilled water was admixed with the collected chloroform phase, and the mixture was allowed to stand to collect the resulting chloroform phase again. The above procedure was sequentially repeated 10 times, and then admixed with an adequate amount of anhydrous magnesium sulfate in usual manner to effect dehydration, concentrated, admixed with 100 parts by weight of saturated aqueous sodium chloride solution, stirred at 60° C. for 30 min, and ice-cooled, followed by removing the supernatant. This procedure was repeated once again. The formed precipitate was redissolved in 300 parts by weight of chloroform, stirred at 60° C. for 30 min, dehydrated with an adequate amount of anhydrous magnesium sulfate in usual manner, and concentrated to obtain an 80% syrup, on a dry solid basis, containing a saccharide having a basic cyclic-structure with methyl group as a substituent. Conventional measurement of ¹H-NMR of the product revealed that it contained methyl group with a mean substitution degree of 7.5. The product can be used in preparing ophthalmic pharmaceutical compositions for treating ophthalmic diseases including cataract, that induce swelling, edema or walleye/cloudiness in the lens and/or the cornea.

The present invention is explained in detail with reference to the following Examples but it should not be restricted thereby:

### EXAMPLE 1

<Ophthalmic solution>
Ingredients in 100 ml;

| Cyclic tetrasaccharide crystal, pentahydrate, | 3.5 | g |
|---|---|---|
| prepared by the method in Example for Reference 2 | | |
| Sodium chloride | 0.4 | g |
| Potassium chloride | 0.15 | g |
| Sodium dihydrogenphosphate | 0.2 | g |
| Borax | 0.15 | g |
| Sterile refined water | q.s. | |
| Total volume | 100 | ml |

The above-identified ingredients were mixed in usual manner and sterilized to obtain a preparation for use as an ophthalmic solution. The pH was adjusted to 7.3. The product can be used for preventing and treating the swelling, edema or walleye/cloudiness in the lens and/or the cornea inducible by ophthalmic diseases such as cataract.

Administration of 0.05 ml/shot of the product to eyes of five rabbits at a frequency of 10 times a day for one month every day caused no abnormalities such as ophthalmic inflammation, hyperemia, and edema, as well as the swelling, edema and walleye/cloudiness in the lens or the cornea, revealing that the product is an ophthalmic solution administrable safely and successively for a relatively long period of time.

### EXAMPLE 2

Ophthalmic solution
Ingredients in 100 ml;

| A syrup, containing cyclic tetrasaccharide | 3.7 | g |
|---|---|---|
| and branched cyclic tetrasaccharide, prepared by | | |
| the method in Example for Reference 1 | | |
| Sodium chloride | 0.4 | g |
| D-Glucose | 0.04 | g |
| Sterile refined water | q.s. | |
| Total volume | 100 | ml |

The above-identified ingredients were mixed in usual manner and sterilized to obtain an ophthalmic solution. The pH was adjusted to 7.2. The product can be used for preventing and treating the swelling, edema or walleye/cloudiness in the lens and/or the cornea inducible by ophthalmic diseases such as cataract.

### EXAMPLE 3

Ophthalmic solution
Ingredients in 100 ml;

| An amorphous powder, containing | 7.0 | g |
|---|---|---|
| cyclic tetrasaccharide and branched | | |
| cyclic tetrasaccharide bound with one | | |
| mole of D-galactose as a substituent, | | |
| prepared by the method in Example for Reference 3 | | |
| D-Glucose | 0.04 | g |
| Sterile refined water | q.s. | |
| Total volume | 100 | ml |

The above-identified ingredients were mixed in usual manner and sterilized to obtain an ophthalmic solution. The pH was adjusted to 7.3. The product can be used for preventing and treating the swelling, edema or walleye/cloudiness in the lens and/or the cornea inducible by ophthalmic diseases such as cataract.

### EXAMPLE 4

Ophthalmic solution
Ingredients in 100 ml;

| A syrup, containing a saccharide | 0.5 | g |
|---|---|---|
| having a basic cyclic-structure | | |
| with methyl group, prepared by the method in | | |
| Example for Reference 4 | | |
| Sodium chloride | 0.6 | g |
| Potassium chloride | 0.15 | g |
| Sodium dihydrogenphosphate | 0.2 | g |
| Borax | 0.15 | g |
| Benzalkonium chloride | 0.005 | g |
| Sterile refined water | q.s. | |
| Total volume | 100 | ml |

The above-identified ingredients were mixed in usual manner and sterilized to obtain an ophthalmic solution. The pH was adjusted to 7.0. The product can be used for preventing and treating the swelling, edema or walleye/cloudiness in the lens and/or the cornea inducible by ophthalmic diseases such as cataract.

### EXAMPLE 5

Ophthalmic solution
Ingredients in 100 ml;

| Cyclic tetrasaccharide, pentahydrate, prepared by | 2.5 | g |
|---|---|---|
| the method in Example for Reference 2 | | |
| Hydrous crystalline α,α-trehalose, produced by | 2.0 | g |
| Hayashibara Biochemical Laboratories, Inc., | | |
| Okayama, Japan | | |
| Sodium chloride | 0.4 | g |
| Potassium chloride | 0.15 | g |
| Sodium dihydrogenphosphate | 0.2 | g |
| Borax | 0.15 | g |
| Ascorbic acid 2-glucoside, a reagent grade | 0.1 | g |
| specimen, commercialized by Hayashibara Biochemical | | |
| Laboratories, Inc., Okayama, Japan | | |
| Sterile refined water | q.s. | |
| Total volume | 100 | ml |

The above-identified ingredients were mixed in usual manner and sterilized to obtain an ophthalmic solution. The pH was adjusted to 7.3. The product can be used for preventing and treating the swelling, edema or walleye/cloudiness in the lens and/or the cornea inducible by ophthalmic diseases such cataract.

### EXAMPLE 6

Ophthalmic wash
Ingredients in 100 ml;

| Cyclic tetrasaccharide, pentahydrate, prepared by | 0.1 | g |
|---|---|---|
| the method in Example for Reference 2 | | |
| Sodium chloride | 0.4 | g |
| Potassium chloride | 0.05 | g |
| Calcium chloride | 0.01 | g |
| Magnesium sulfate | 0.01 | g |
| Sodium citrate | 0.05 | g |
| Sodium hydrogencarbonate | 0.2 | g |
| Maltose | 0.15 | g |
| 1N-Hydrochloric acid | q.s. | |
| Sterile refined water | q.s. | |
| Total volume | 100 | ml |

The above-identified ingredients were mixed in usual manner and sterilized to obtain an ophthalmic solution. The pH was adjusted to 7.2. The product can be used for preventing and treating the swelling, edema or walleye/cloudiness in the lens and/or the cornea inducible by ophthalmic diseases such as cataract.

### EXAMPLE 7

Ophthalmic ointment
Ingredients in 100 g;

| A syrup, containing cyclic tetrasaccharide, | 4.5 | g |
|---|---|---|
| prepared by the method in Example for Reference 1 | | |
| Liquid paraffin | 5.0 | g |
| White petrolatum | q.s. | |
| Total volume | 100 | ml |

The above-identified ingredients were mixed in usual manner and sterilized to obtain an ophthalmic solution. The product can be used for preventing and treating the swelling, edema or walleye/cloudiness in the lens and/or the cornea inducible by ophthalmic diseases such as cataract.

### EXAMPLE 8

Ophthalmic injection
Ingredients in 100 g;

| Cyclic tetrasaccharide, pentahydrate, | 2 | g |
|---|---|---|
| prepared by the method in Example for Reference 2 | | |
| Physiological saline | q.s. | |
| Total volume | 100 | ml |

The above-identified ingredients were mixed in usual manner and sterilized, and five gram aliquots of the resulting solution were distributed into vials and lyophilized into an ophthalmic injection. The product can be used for preventing and treating the swelling, edema or walleye/cloudiness in the lens and/or the cornea inducible by ophthalmic diseases such as cataract.

### INDUSTRIAL APPLICABILITY

The saccharide having a basic cyclic-structure has improved therapeutic and/or prophylactic effects on the swelling, edema and walleye/cloudiness in the lens and/or the cornea induced by cataract, etc. Also, since the saccharide having a basic cyclic-structure is a safe and stable saccharide, the ophthalmic pharmaceutical composition containing the same can be successively used safely for a relatively long period of time without fear of causing side effect. The ophthalmic pharmaceutical composition containing the saccharide having a basic cyclic-structure according to the present invention shows a distinct effect on the improvement of the symptoms of swelling, edema and walleye/cloudiness in the lens and/or the cornea, and thus it can be advantageously used in the treatment and/or prevention of such symptoms. Further, since the saccharide having a basic cyclic-structure is a safe and stable saccharide, it can be successively used safely for a relatively long period of time without fear of causing side effect. The present invention with such outstanding functions and effects is a significant invention that greatly contributes to this art.

1. A method for treating the swelling, edema and/or walleye/cloudiness in the lens and/or the cornea, which comprises a step of administering a composition to a subject's eye(s), said composition consisting of i) a saccharide having a basic structure represented by General Formula 1, ii) water, and iii) one or more members selected from the group consisting of maltose, sodium chloride, potassium chloride, sodium dihydrogenphosphate, calcium chloride, magnesium chloride, sodium citrate, sodium hydrogencarbonate, borax, D-glucose, benzalkonium chloride, α,α-trehalose, ascorbic acid 2-glucoside, hydrochloric acid, liquid paraffin, white petrolatum, physiological saline, and magnesium sulfate, wherein the content of said saccharide i) to the total saccharide content in said composition is 98% or higher, said composition being in the form of a member selected from the group consisting of ophthalmic solution, ophthalmic wash, ophthalmic ointment, and injection, wherein the amount of said saccharide i) in said composition is about 0.1 to 20% to the total weight of said composition, thereby treating the swelling, edema and/or walleye/cloudiness in the lens and/or the cornea: 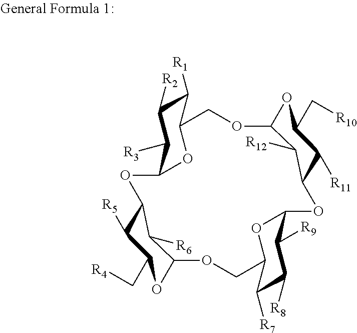
where, R₁ through R₁₂ independently represent a substituent selected from the group consisting of hydroxyl group, O-glycosyl group, and O-alkyl group.

2. The method of claim 1, wherein said saccharide having a basic structure represented by General Formula 1 is a cyclic tetrasaccharide having a cyclic structure of cyclo{→6)-α-D-glucopyranosyl-(1→3)-α-D-glucopyranosyl-(1→6)-α-D-glucopyranosyl-(1→3)-α-D-glucopyranosyl-(1→}.

3. The method of claim 1, wherein the swelling, edema and/or walleye/cloudiness in the lens and/or the cornea are induced by cataract.

4. A method for treating the swelling, edema and/or walleye/cloudiness in the lens and/or the cornea, which comprises a step of administering a composition to a subject's eye(s), said composition consisting of i) a saccharide having a basic structure represented by General Formula 1, ii) water, and iii) one or more members selected from the group consisting of maltose, sodium chloride, potassium chloride, sodium dihydrogenphosphate, calcium chloride, magnesium chloride, sodium citrate, sodium hydrogencarbonate, borax, D-glucose, benzalkonium chloride, α,α-trehalose, ascorbic acid 2-glucoside, hydrochloric acid, liquid paraffin, white petrolatum, physiological saline, and magnesium sulfate, wherein the content of said saccharide i) to the total saccharide content in said composition is 98% or higher, said composition being in the form of a member selected from the group consisting of ophthalmic solution, ophthalmic wash, ophthalmic ointment, and injection, wherein the amount of said saccharide i) in said composition is at least 0.01% by weight to the total weight of said composition, thereby treating the swelling, edema and/or walleye/cloudiness in the lens and/or the cornea: 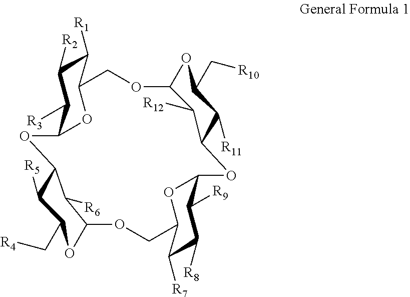
where, R₁ through R₁₂ independently represent a substituent selected from the group consisting of hydroxyl group, O-glycosyl group, and O-alkyl group.

5. A method for treating the swelling, edema and/or walleye/cloudiness in the lens and/or the cornea, which comprises a step of administering a composition to a subject's eye(s), said composition consisting of i) a saccharide having a basic structure represented by General Formula 1, ii) water, and iii) one or more members selected from the group consisting of maltose, sodium chloride, potassium chloride, sodium dihydrogenphosphate, calcium chloride, magnesium chloride, sodium citrate, sodium hydrogencarbonate, borax, D-glucose, benzalkonium chloride, α,α-trehalose, ascorbic acid 2-glucoside, hydrochloric acid, liquid paraffin, white petrolatum, physiological saline, and magnesium sulfate, wherein the content of said saccharide i) to the total saccharide content in the composition is 98% or higher; iv) and at least one optional ingredients selected from the group consisting of alcohols, p-oxymethyl benzoate, sodium dehydroacetate, buffers, thickeners, solubilizing agents, stabilizers, bases for ophthalmological ointment, and mixtures thereof, said composition being in the form of a member selected from the group consisting of ophthalmic solution, ophthalmic wash, ophthalmic ointment, and injection; and wherein the amount of said saccharide i) in said composition is about 0.1 to 20% to the total weight of said composition, thereby treating the swelling, edema and/or walleye/cloudiness in the lens and/or the cornea: 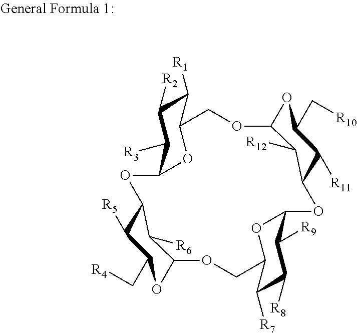
where, R₁ through R₁₂ independently represent a substituent selected from the group consisting of hydroxyl group, O-glycosyl group, and O-alkyl group.

6. The method according to claim 5, wherein the buffer is boric acid; the thickeners are selected from the group consisting of methyl cellulose, carboxymethyl cellulose, chondroitin sulfate, polyvinyl alcohol, and pullulan; the stabilizing agents are polysorbate 80; and the stabilizers are selected from the group consisting of sodium edetate and sodium hydrogensulfite.

7. A method for treating the swelling, edema and/or walleye/cloudiness of the lens and/or the cornea, which comprises a step of administering a composition to a subject's eye(s), said composition consisting of:
i) a saccharide having a basic structure represented by General Formula 1,
ii) water; and
iii) one or more members selected from the group consisting of maltose, sodium chloride, potassium chloride, sodium dihydrogenphosphate, calcium chloride, magnesium chloride, sodium citrate, sodium hydrogencarbonate, borax, D-glucose, sterile refined water, benzalkonium chloride, α,α-trehalose, ascorbic acid 2-glucoside, hydrochloric acid, liquid paraffin, white petrolatum, physiological saline, and magnesium sulfate, wherein the content of said saccharide i) to the total saccharide content in said composition is 98% or higher, said composition being in the form of a member selected from the group consisting of intraocular infusion solution, wash for anterior chamber, internal medicine, or preservative for extracted cornea; and wherein the amount of said saccharide i) in said composition is about 0.1 to 20% to the total weight of said composition, thereby treating the swelling, edema and/or walleye/cloudiness in the lens and/or the cornea: 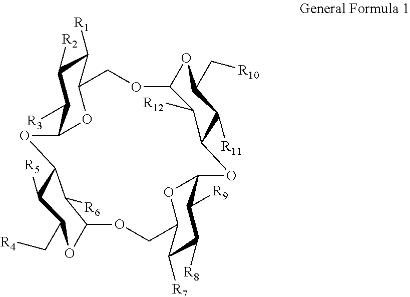
where, R₁ through R₁₂ independently represent a substituent selected from the group consisting of hydroxyl group, O-glycosyl group, and O-alkyl group.
